(19) United States
(12) Patent Application Publication
(10) Pub. No.: US 2018/0125416 A1
(43) Pub. Date: May 10, 2018

(54) APPARATUS AND METHOD FOR TREATMENT OF BIOLOGICAL STRUCTURE

(71) Applicant: BTL HOLDINGS LIMITED, Limassol (CY)

(72) Inventors: Tomás Schwarz, Prague (CZ); Ondra Prouza, Ricany u Prahy (CZ)

(21) Appl. No.: 15/344,811

(22) Filed: Nov. 7, 2016

(51) Int. Cl. A61B 5/00; A61B 5/11; A61G 15/00; A61N 2/00; A61N 2/02

(52) U.S. Cl. CPC A61B 5/486; A61B 5/0077; A61B 5/1128; A61B 2090/064; A61N 2/004; A61N 2/02; A61G 15/007

(57) ABSTRACT

A magnetic stimulation device has components for positioning a patient into a correct treatment position, and for improving patient comfort. The methods may be used for improving the effectiveness of the stimulation by setting a magnetic field generating device in appropriate position by using feedback information.

### FIELD OF THE INVENTION

The present invention relates to an apparatus and method for treating a patient by a magnetic field. The application of the magnetic field is provided by at least one high power magnetic field generating device.

### BACKGROUND OF THE INVENTION

Magnet therapy uses the influence of magnetic flux on biological tissue. Electric current is induced in the tissue due to voltage change which causes a polarization of the cell membrane. A fundamental phenomenon of electric current in biological tissue is a transfer of neural excitation or muscle contraction. The intensity of the effect is dependent on the magnetic flux density, repetition rate of the pulses, impulse time duration or envelope of the stimulation signal. One possible application of magnetic treatment is treatment of urogenital diseases, e.g. incontinence or pain in the pelvic area.

Presently magnet treatment is widely applied for treatment of urinary incontinence. The currently used treatment devices are in the form of chair with an integrated coil beneath the seating portion. The coil is fixed within the treatment device and its position is static. A magnetic field generated by the coil is intensified and/or focused by using of magnetic core elements of various shapes, e.g. U-shaped or J-shape type core elements.

The coil is integrally fixed in the seating portion of the chair. A plurality of the J-shape magnetic core elements are surrounded by the coil generating a pulsed magnetic field. The end portions of the J-shape magnetic core elements are within proximity of patient's anus and urethra and/or genital area, e.g. vagina. The magnetic flux is delivered to the patient by the magnetic core elements. Two or four magnetic core elements are most frequently used. The treatment chair also may consist of at least one U-shape magnetic core element with at least one coil wound around the end portions of the magnetic core element. The magnetic field stimulates muscles of pelvic floor via stimulation of pudendal nerves.

The patient sits on the seating portion ergonomically formed to be comfortable for the patient. The chair also includes armrests and backrest for improving patient's comfort. The patient is relaxed in the chair.

The mutual orientation of rotating plates may also be manually adjustable via thumbscrews to fit the coils to the patient region of urethra opening and clitoris. The orientation of the coils is adjusted prior to treatment to stimulate mainly pudendal nerves.

The present devices lack the possibility of dynamic adjusting of the orientation and/or position of the magnetic field generating device to focus or defocus the peak of magnetic treatment to stimulate small or large target biological structure, and are not designed for automatic operation.

Additionally, more efficient treatment devices are needed. There is also a need for improvements for allowing a patient to be positioned in a correct treatment position and to feel comfortable.

Existing treatment devices also lack feedback devices for adjusting the treatment parameters and/or the orientation of the magnetic field generating device during the treatment dynamically.

### SUMMARY OF THE INVENTION

A magnetic stimulation device may include at least one adjustable component for positioning the patient into a correct treatment position to provide improved effectiveness of the treatment. The magnetic stimulation device may provide improved results by the combined effects of correct treatment position of the patient and the magnetic treatment.

A movable magnetic field generating device may be used, including apparatus for adjusting a position and/or orientation of the magnetic field generating device.

A feedback system may be used to provide feedback information to the magnetic stimulation device to improve the effectiveness of the treatment. The treatment parameters may be influenced by the feedback to improve the treatment. The feedback also provides for adjusting the position and/or orientation of the magnetic field generating device if the patient is repositioned from the correct treatment position.

Pretreatment sequences improve the magnetic treatment by positioning the patient into the correct treatment position and/or by determining at least one treatment parameter according to the patient's needs. The pretreatment sequences enable self-operated treatment.

### BRIEF DESCRIPTION OF THE DRAWINGS

FIGS. 1a and 1b illustrate circuits for providing magnetic pulses.

FIG. 2 illustrates a magnetic stimulation device.

FIGS. 3a and 3b illustrate exemplary embodiments of patient supporting apparatus.

FIG. 4 illustrates a setting of external applicators in a seating portion of a magnetic stimulation device.

FIGS. 5a-5c illustrate an exemplary locking mechanism.

FIG. 6 illustrates an exemplary embodiment for adjusting the magnetic field generating device.

FIG. 7A illustrates details of an exemplary embodiment for focusing the magnetic field.

FIG. 7B illustrates an exemplary embodiment for focusing the magnetic field.

FIG. 8 illustrates an exemplary kinematic scheme of a positioning mechanism.

FIG. 9 illustrates an exemplary embodiment of a positioning mechanism.

FIG. 10 illustrates an exemplary embodiment of a positioning mechanism.

FIG. 11 illustrates an exemplary embodiment of a positioning mechanism.

FIG. 12 illustrates an exemplary embodiment of feedback.

FIG. 13 is a flow chart of an algorithm used in a self-operated magnetic stimulation device.

FIG. 14 illustrates an exemplary embodiment of a pressure sensitive layer.

FIGS. 15a and 15b illustrate exemplary embodiments of pressure sensitive layers.

### GLOSSARY

Biological structure includes a cell, a neuron, a nerve, a muscle fiber, a muscle, a tissue or a ligament.

Stimulation refers to a magnetic flux density inducing an electric current in the biological structure.

Impulse refers to the only one magnetic stimulus.

Pulse refers to a period of stimulation signal of at least one magnetic stimulus and time duration of no stimulation, i.e. time duration between two impulses from rise/fall edge to next rise/fall edge.

Repetition rate refers to frequency of firing the pulses; it is derived from the time duration of a pulse.

Correct treatment position refers to the patient's position in which the treatment is the most effective compared to any other patient's position using the same treatment parameters.

Treatment parameters refer to magnetic flux density, repetition rate, impulse duration, treatment duration, position and/or orientation of the magnetic field generating device.

Active response refers to any biological reaction influenced by the stimulation by time-varying magnetic field including e.g. a change in a permeability of cell membrane for ions or any other particles, a generation of an action potential or at least partial muscle contraction.

Appropriate position refers to the position of the magnetic field generating device where the ideal biological response is induced by stimulation with time-varying magnetic field.

Ideal biological response refers to active response induced by stimulation of e.g. a muscle motor point or the weakest biological response.

Motor point refers to a small region of a muscle in which motor endplates are aggregated i.e. the muscle is most sensitive to stimulation by time-varying magnetic field at this point.

### DETAILED DESCRIPTION

Electric current is induced in the stimulated biological structure during time-varying magnet treatment. A distribution of magnetic field is uniform in the biological structure. Particles (e.g. atoms, ions, molecules etc.) in the biological structures are influenced by the magnetic field and permeability of a cell membrane also increases.

The present methods may be used for treatment of disease of urogenital and/or digestive tract, e.g. improvement of circulation and/or trophic problems, faecal incontinence, urinal incontinence (stress or urge), neuromuscular dysfunction of bladder, mixed incontinence, sexual dysfunction, priapism, erectile dysfunction, orgasmic disorder, fertility issues, chronic pelvic pain syndrome, pain in pelvic area, hyperplasia of prostate, prostatitis, prostatodynia syndrome, dysmenorrhea, vulvodynia, pain and other conditions associated with menstrual cycle, menopausal and/or postmenopausal disorders, cystitis (such as interstitial), inflammatory disease of uterus or cervix uteri, parametris, peritonitis, vaginitis, vulvitis, endometriosis, genital prolapse, hemorrhoids, peripheral paresis or pelvic floor issues in general. The present methods may be used for muscle strengthening, muscle relaxation, regeneration after childbirth (such as pelvic floor prolapse), vaginal tightening or scar treating.

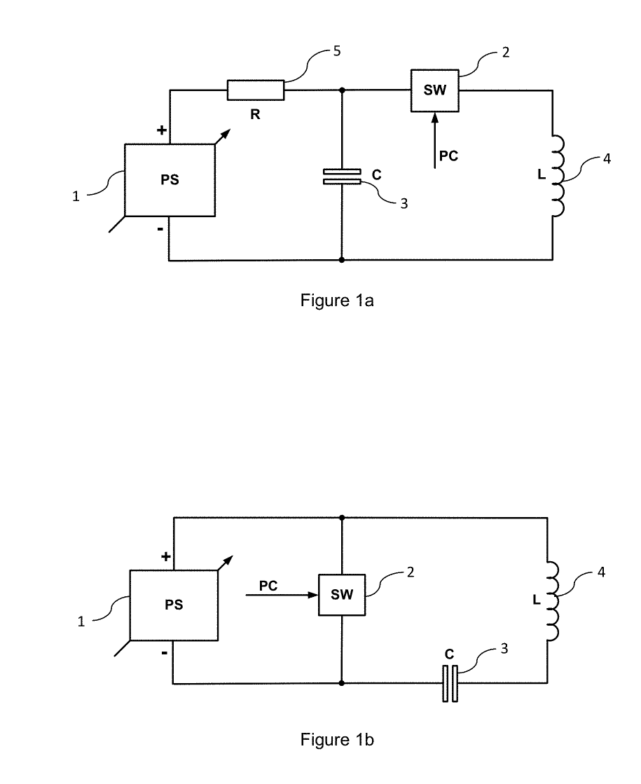

The present invention relates to apparatus and methods of operating said apparatus for stimulation of biological structure by time-varying magnetic field of magnetic flux density sufficient to induce at least partial muscle contraction. Referring to FIGS. 1a and 1b, a circuit for generating magnetic pulses includes a connection to energy source 1, a switching device 2, an energy storage device 3 and a magnetic field generating device 4. Alternatively the magnetic stimulation device may include protective resistors and/or protective circuitry 5. In the preferred embodiment the switching device 2 is connected in parallel to the magnetic field generating device 4 and the energy storage device 3, and the energy storage device 3 is in series with the magnetic field generating device 4 as illustrated in FIG. 1b. Broad spectrum of applications of biological structure stimulation by time-varying magnetic field is achieved due to high repetition rates and/or high value of magnetic flux density.

The present method stimulates the biological structure, preferably at least one pelvic floor muscle, by pulsed magnetic field defined by peak to peak magnetic flux density of at least 0.1 T, more preferably at least 0.5 T, even more preferably at least 1 T, even more preferably at least 1.5 T, most preferably at least 2 T, or up to 7 Tesla on the coil surface and/or repetition rate of at least 100, 120, 140, 180, 200, 250 or up to 700 Hertz with treatment/successive treatments lasting several seconds or longer, e.g. at least 5, 10, 30, 60, 120 or 240 seconds, or longer. The impulse width is in the range of tens to hundreds of μs.

The magnetic stimulation device may include at least one component improving ergonomics and/or patient comfort during the treatment. The component may be e.g. a seating portion, back rest, arm rest, adjustable front resting apparatus or patient supporting apparatus sufficiently maintaining the patient in a sitting position. The effectiveness of the treatment is maximal in the correct treatment position compared to any other position using the same treatment parameters because the target biological structure is within the closest proximity of the magnetic field generating device, e.g. a coil. The correct treatment position of the patient may provide improved treatment effects in combination with magnet treatment. Additionally the muscles of pelvic floor may be activated in the correct treatment position. The activation of the pelvic floor muscles may be caused by the position of the patient's torso with respect to vertical direction. Hence the treatment is improved by positioning the patient in the correct treatment position and maintaining the patient in the correct treatment position with appropriate comfort for the patient.

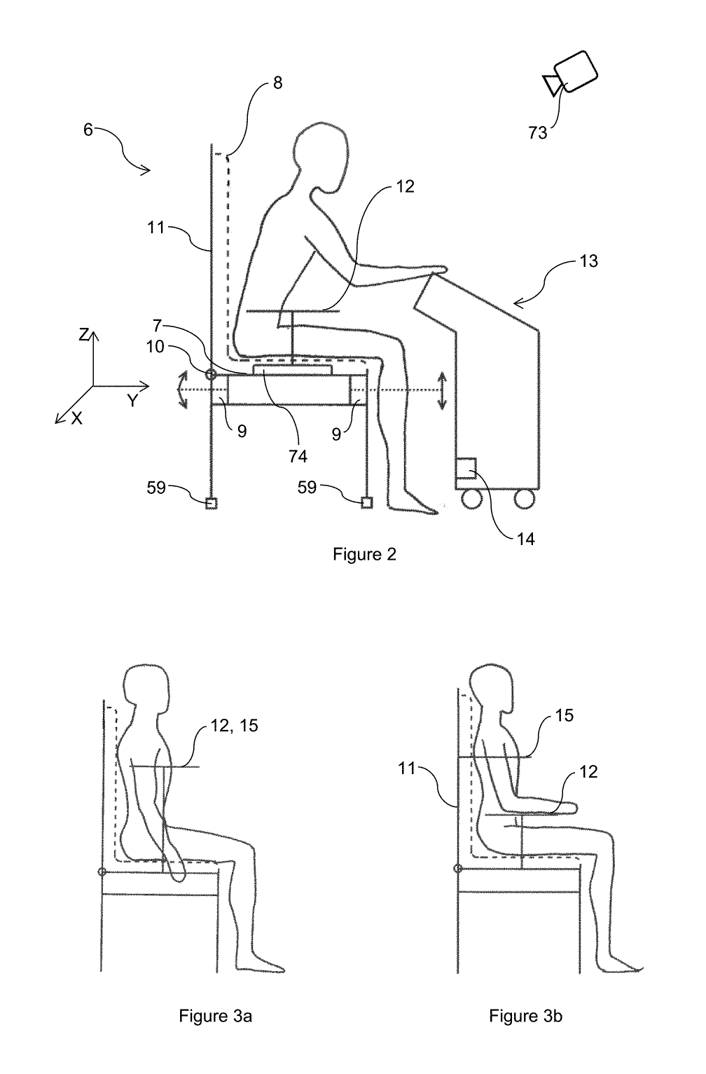

FIG. 2 illustrates the magnetic stimulation device 6 including a seating portion 7 for providing more effective treatment. The seating portion may be adapted to fit the patient's buttocks. The shape of the seating portion 7 may be e.g. a circle, an oval, a square or a rectangle. The seating portion 7 may be extendable following the patient needs. Hence the patient's comfort is provided at a high level and the correct treatment position may be maintained for the entire period of treatment time. In an alternative aspect the seating portion 7 may include a comfortable cover 8 which may be integral or detachable to the seating portion. The cover 8 may be e.g. cushion made of gelatinous material or memory foam, active pressure redistribution cushion, low-air-loss cushion or cushion maintaining low pressure of fluid media. All the cushion types may be changeable and/or removable to be cleaned to provide high hygiene standard for the patient.

The seating portion may be movable, e.g. in at least one axis of Cartesian coordinate system (CCS). The seating portion 7 may be moved by at least one seat actuator 9 in rotational and/or translational movement, i.e. the seating portion may be tilted about a pivot axis 10 (corresponding to X-axis of CCS) or shifted, e.g. in a direction corresponding to X, Y and/or Z axes of CCS. The movement may set the patient into a correct treatment position and maintain the patient in the correct treatment position. Alternatively the movement of the seating portion 7 may be used for dynamic positioning of the patient to mechanically induce the muscle contraction in response to mechanical movement of the seating portion 7. The muscle contraction may be induced by e.g. vibrational movement of the seating portion 7. Alternatively the movement of the seating portion may be used for positioning of the patient suffering from any muscle imbalance of patient's torso and/or any incorrect body posture. The seat actuator may include motors or actuators and linkages to provide movement of the seating portion. Alternatively the seat actuator 9 may be manually operated to move the seating portion into a desired position.

The magnetic stimulation device 6 may include a back rest 11 for maintaining the patient in a correct treatment position and providing comfort for the patient during the treatment. The back rest 11 may be adjustable following the patient's anatomical needs, e.g. the back rest 11 may be adjustable in its length, height (adjustment in Z-axis of CCS) and/or the inclination (rotation around X-axis of CCS). The inclination may be preferably adjusted by movement of the back rest 11 around pivot axis 10. The back rest 11 may be extendable as well. In an alternative embodiment the back rest 11 may include movable parts for massaging the patient's back, e.g. rollers. In another alternative embodiment at least one arm rest may be detachable or integral part of the back rest 11.

The magnetic stimulation device 6 may include at least one arm rest 12 for maintaining the patient in correct treatment position and providing high comfort level for the patient during the treatment. The at least one arm rest 12 may be adjustable following the patient's anatomical needs. Arm rest 12 may be adjustable with reference to seating portion 7 of the magnetic stimulation device 6, e.g. it may be extendable, height adjustable and/or adjustable by rotation around X and/or Z axes of CCS. In the preferred embodiment the adjustment of at least one arm rest 12 is independent. In an alternative embodiment the adjustment of each arm rest may be dependent, e.g. the arm rests may be linked via a mechanism.

The magnetic stimulation device 6 may include a resting apparatus 13 for maintaining the patient in a slightly bent or reclined position. The inclination of patient's torso with respect to vertical direction may be in the range of −90 to 90°, more preferably in the range of −45 to 45°, most preferably in the range of −30 to 30°. The tilting portion of the resting apparatus 13 may be adjustable in angle and/or in height. The resting apparatus 13 may be also side adjustable. A distance of the resting apparatus 13 from the magnetic stimulation device 6 may be adjustable as well. The resting apparatus 13 may be preferably situated in front of the magnetic stimulation device 6. In the preferable embodiment the patient may be in contact with the resting apparatus 13 by hand or forearm. In an alternative embodiment the patient may lean against the resting apparatus 13 by chest or any part of upper extremity such as arm or armpit.

In an alternative embodiment the resting apparatus may be represented as an adjustable belt. The belt may be detachably attached to the back rest.

In an alternative embodiment the position of the resting apparatus, e.g. an inclination or a distance from the magnetic stimulation device 6, may be tracked by a sensor 14 to obtain feedback information to adjust treatment parameters to provide the most efficient treatment to the patient. It is clear to a person skilled in the art which sensor is suitable for such a purpose and how to use the at least one sensor for the purpose. In the preferred embodiment such sensor may be any kind of an inclinometer, an accelerometer, a load cell, a force, a magnetic, a distance or an optic sensor. Alternatively, the feedback information may be used for safety reasons, e.g. notification of a safe position may be provided by the magnetic stimulation device and/or resting apparatus when the resting apparatus is within a predetermined distance limit from the magnetic stimulation device. The distance limit may be adjusted by an operator. Alternatively notification of a less safe position may be provided if the distance between magnetic stimulation device and the resting apparatus exceeds the predetermined distance limit. The notification may be in human perceptible form e.g. by mechanical and/or electromagnetic apparatus, such as audibly perceptible notification (e.g. beep) or visually perceptible notification (flashing light, color change etc.).

The magnetic stimulation device may include a patient supporting apparatus for maintaining the patient in correct treatment position and providing high comfort level for the patient during the treatment in the case that the patient is e.g. spinal patient, paralyzed or plegic patient. The patient supporting apparatus may at least partially bear the weight of the patient. The patient supporting apparatus may be adjustable following the patient's anatomical needs. Patient supporting apparatus may be adjustable with reference to seating portion 7 of the magnetic stimulation device 6, e.g. it may be extendable, height adjustable and/or adjustable by rotation around X and/or Z axes of CCS. The patient supporting apparatus may raise or lower the patient, or the torso and/or limbs of the patient, or otherwise maintain the patient in the correct treatment position. FIG. 3a illustrates the preferred embodiment of the patient supporting apparatus 15 which may be high adjustable arm rest 12 and/or back rest. FIG. 3b illustrates an alternative embodiment of the patient supporting apparatus 15 which may be detachable or integral part of the back rest 11. In still another embodiment the patient supporting apparatus 15 may be separate part of the magnetic stimulation device. In FIGS. 3a and 3b the patient is maintained in the correct treatment position by the patient supporting apparatus contacting the patient's armpits.

The patient may be positioned by any part of the magnetic stimulation device to correct treatment position. The correct treatment position may be preferably one position during the treatment. In an alternative embodiment the correct treatment position may vary during the treatment hence the treatment position may be dynamically changed following the stimulated target biological structure. The position of the patient may be adjusted manually and/or automatically via at least one actuator. The actuator may preferably tilt the seating portion, back rest or both. Various types of positioning mechanisms may be used for adjusting the position of the patient, e.g. rotational, translational or complex mechanism such as roll-slide mechanism.

All the resting parts such as back rest, arm rests, adjustable resting apparatus may be separate, integral or detachable to the magnetic stimulation device. The apparatus for attaching the resting parts to the magnetic stimulation device may be represented by various embodiments. All contact surfaces of the magnetic stimulation device may be bolstered by soft material. All contact surfaces may be preferably made of well-cleanable material to provide the patient high hygiene standard, in an alternative embodiment the contact surface may be made of sterilizable material. In an alternative embodiment the soft material may be covered by disposable cover.

The magnetic stimulation device may include a plurality of magnetic field generating devices. The positions of the at least two magnetic field generating devices may focus the magnetic fields to the target area; or the magnetic field generated by one magnetic field generating device may interfere with the magnetic field generated by another magnetic field generating device and the resulting magnetic field may be shaped. The magnetic flux density may be summed from the plurality of magnetic field generating devices.

The plurality of the magnetic field generating devices may extend the active time duration of the stimulation in the case that the switching devices are switched in sequence. Therefore the treatment is more effective and the treatment time may be shortened.

The plurality of magnetic field generating devices may be used for treating at least two cooperating muscle groups. In an exemplary application one muscle may be treated to achieve myostimulation effect and other muscle may be treated to achieve myorelaxation effect, analgesic effect may be alternatively induced. Alternatively at least two different muscles or muscle layers may be stimulated by with the same effect.

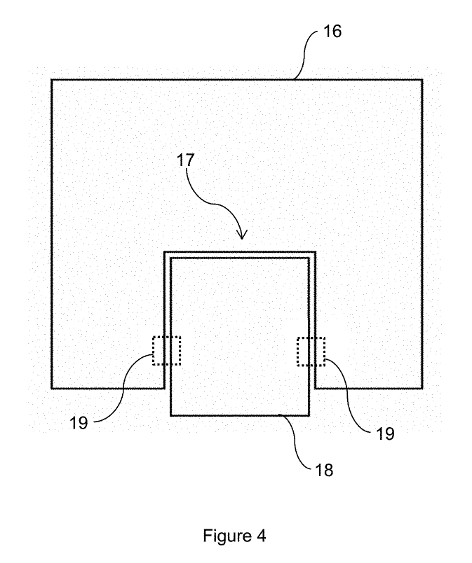

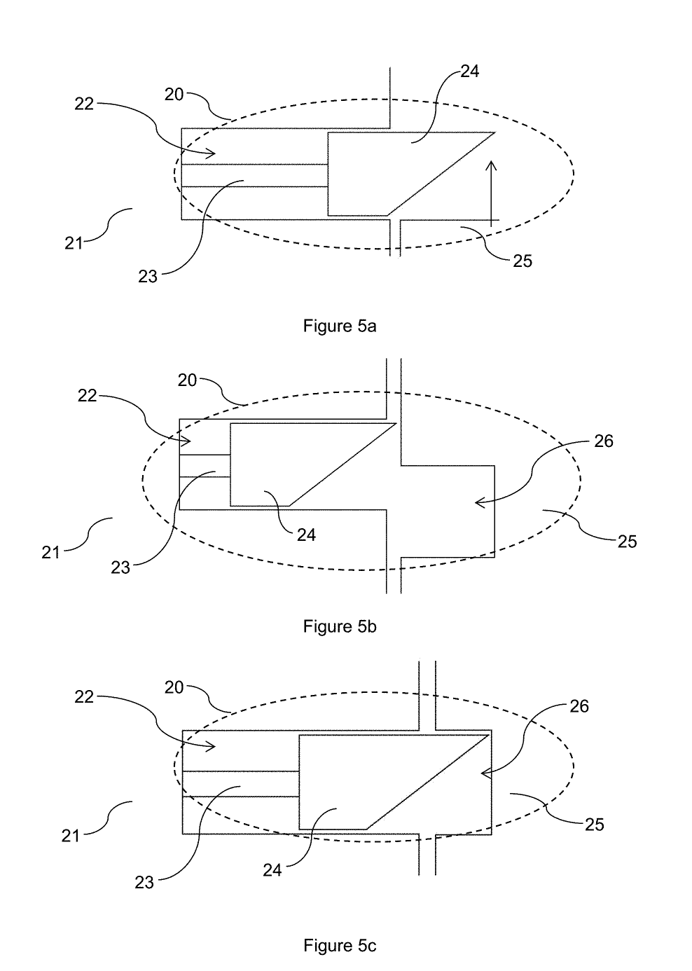

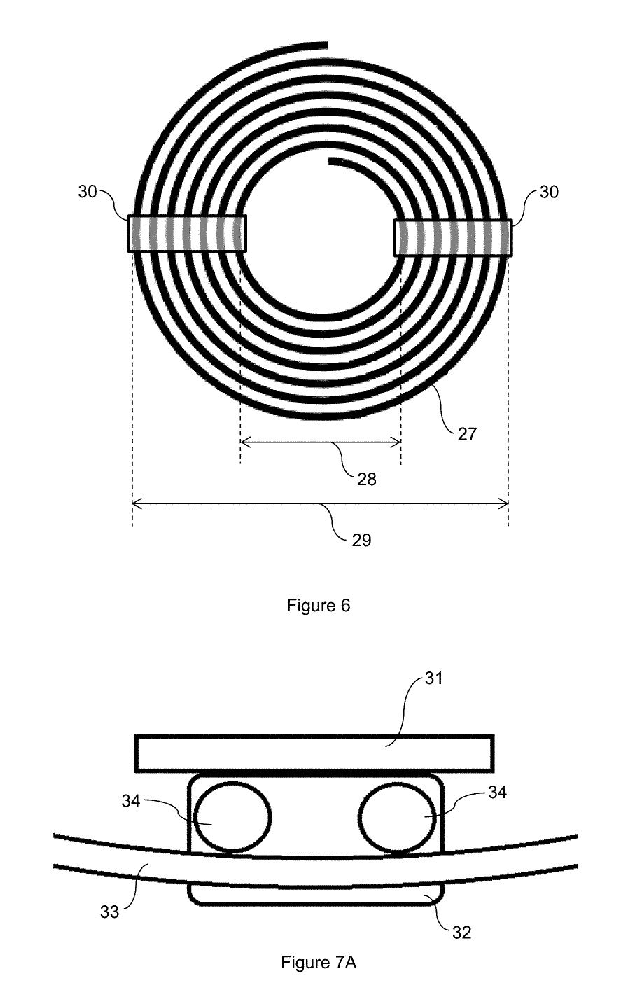

Alternatively the magnetic field generating device may be in an external applicator such as hand-held applicator. FIG. 4 illustrates the seating portion 16 of the magnetic stimulation device including a recess 17 where the external applicator 18 may be attached. The external applicator 18 may be attached preferably into the recess 17 of the seating portion 16 by at least one locking mechanism 19. Preferably a plurality of the locking mechanisms 19 may be used. At least one locking mechanism 19 on at least one side of the external applicator 18 may be used. Preferably at least one locking mechanism 19 on at least two sides of external applicator 18 may be used. An exemplary embodiment of locking mechanism 19 is described in FIGS. 5A-5C which shows a locking mechanism 20 including two cooperating parts. One part is housed in a seating portion 21 and includes a recess 22 of the locking mechanism 20 in seating portion 21, a resilient member 23 and a latching member 24 movable within the recess 22 in the seating portion 21. The second part is housed in the external applicator 25 and includes a recess 26 in the external applicator 25.

FIG. 5a illustrates the exemplary embodiment of the locking mechanism 20 in an unlocked position. The resilient member 23 is relaxed and the latching member 24 is in the extended position where the external applicator 18 doesn't influence the latching member 24. Forward movement of the external applicator 25, which is illustrated by an arrow, moves the latching member 24 to the recess 22 of the locking mechanism 20 in seating portion 21. The movement of the latching member 24 presses the resilient member 23. The resilient member may be made of any material enabling elastic deformation, e.g. rubber or composite. The resilient member may be preferably a spring.

FIG. 5b illustrates the exemplary embodiment of the locking mechanism 20 still unlocked. The latching member 24 is moved by partial insertion of the external applicator 25 into the recess 17 of the seating portion 16 (illustrated in FIG. 4). The latching member 24 is completely pushed by the external applicator 25 to the recess 22 of the locking mechanism 20 in seating portion 21. The resilient member 23 is maximally compressed and the latching member 24 is in a fully withdrawn position.

FIG. 5c illustrates the exemplary embodiment of the locking mechanism 20 in a locked position. The external applicator 25 is in a correct position to be locked. The latching member 24 is forced by the compressed resilient member 23 into the recess 26 in the external applicator 25. The latching member 24 is in recess 26 of the external applicator 25 and in the recess 22 of the locking mechanism 20 in seating portion 21.

Alternatively the external applicator may be guided via guiding mechanisms on both sides of the external applicator and one locking mechanism may be on the front side of the applicator (the front side of the applicator is the side closest to the center of the seating portion). The guiding mechanism may be any kind enabling insertion of the external applicator. The locking mechanism may be a clip type mechanism.

Alternatively the latching member may be circular and the locking movement may be rotatable. The rotatable movement may be biased by a resilient member.

Alternatively the external applicator may be inserted into the seating portion of the magnetic stimulation device. The external applicator may be moveable within the seating portion. The movement of the external applicator within the seating portion of the magnetic stimulation device may be translational and/or rotational according to at least one axis of CCS, more preferably according to at least two axes of CCS, most preferably according to all three axes of CCS. The external applicator may be removably attached to a positioning mechanism described below or the external applicator may be attached a rod enabling movement of the external applicator within the seating portion. The movement of the external applicator within the seating portion of the magnetic stimulation device may be automatic and/or manual.

Alternatively the external applicator may be attached to the seating portion from the below. The locking mechanism may be placed on the lower side of the seating portion to prevent free detaching of the external applicator of the seating portion by gravitational force. The movement of the latching mechanism may be rotational and/or translational.

Alternatively the locking mechanism may be a pin-type mechanism wherein at least one pin moves into a corresponding recess. The movement of the at least one pin may be rotational and/or translational. Preferably a plurality of pins may be used. The pins may be oriented to each other, e.g. at least two pins may be oriented on opposite sides of the external applicator, more preferably at least three pins may be uniformly distributed on a periphery of the external applicator, most preferably at least four pins may be uniformly distributed on a periphery of the external applicator such as at cross positions in the case of an applicator of regular shape.

Alternatively the external applicator may be inserted into the hollow core center recess of the seating portion to be covered by the cover.

Alternatively the external applicator may be inserted into a pocket fixed on the lower side of the seating portion.

All the locking mechanisms may be preferably self-locking and may be unlocked manually by direct operating of the latching member or by any mechanism, e.g. lever, press button actuated or pulling mechanism.

The magnetic stimulation device may include at least one component improving effectiveness and/or shortening the duration of the treatment. The effectiveness of the treatment is maximal in the correct treatment position comparing to any other position using the same treatment parameters because the target biological structure is within the closest proximity of the magnetic field generating device.

The magnetic stimulation device may adjust the position and/or orientation of the magnetic field generating device with respect to the patient. The position and/or orientation of the magnetic field generating device may be set statically before the treatment to focus the target biological structure to be stimulated the most efficiently. In an alternative embodiment the position and/or orientation of the magnetic field generating device may be adjusted dynamically during the treatment to treat the target biological structure from different direction with the static focus point while stimulating different surface structures. This approach may be useful for selective stimulation of deep muscle structures, i.e. a muscle partially covered by superficial muscle.

The magnetic field may be focused by interference of the magnetic fields generated by a plurality of magnetic field generating devices and/or by adjusting at least one dimension of the magnetic field generating device.

FIG. 6 illustrates an exemplary embodiment providing adjusting of at least one dimension of magnetic field generating device 27, e.g. inner diameter 28 and/or outer diameter 29 of the magnetic field generating device. The at least one dimension of the magnetic field generating device may be adjusted by movement of at least one shape adjusting member 30.

FIG. 7a illustrates a cross-sectional view of an exemplary embodiment for focusing the magnetic field for treatment. The magnetic field generating device 31 is attached to a moveable member 32 which movement corresponds with the shape of guiding member 33. The guiding member 33 may be designed to guide the movement of the at least one rotating member 34 enabling movement of the moveable member 32. The moveable member may be made of rigid material to constitute a housing for at least one rotating member 34, e.g. ball or cylinder. In the preferred embodiment a bearing may be used as the moveable member. Alternatively the moveable member may slide according to guiding member itself without any rotating member. The guiding member profile may be preferably fit to the rotating member. In an exemplary embodiment the guiding member 33 is a rail for guiding the rotating movement of the rotating members 34. However, the guiding member may be formed in various shapes which correspond with the predetermined movement of the magnetic field generating device, e.g. circular shape may be used for focusing the magnetic field to a circle center.

The focusing of the magnetic field may be enabled by a movement of the magnetic field generating device. FIG. 7b illustrates an exemplary embodiment for focusing the magnetic field including a semicircular guiding member 100 for guiding the movement of the magnetic field generating device 101. The magnetic field generating device 101 is moveable according to guiding member 100 (movement is illustrated by arrows). The movement of the magnetic field generating device from the center position (illustrated in solid lines) to extreme positions (illustrated by dotted lines) may create a focus point 103 of the generated magnetic field 102. The focus point may be a biological structure which is stimulated the longest during the treatment.

Alternatively a person skilled in the art may focus the magnetic field by various approaches.

The magnetic stimulation device may adjust the position and/or orientation of the magnetic field generating device with respect to the patient dynamically during the treatment. Dynamic movement of the magnetic field generating device may move the focus point of the stimulation to stimulate larger areas and/or volumes of the target biological structure, e.g. large muscles or a plurality of muscles.

The movement of the at least one magnetic field generating device may be constant or accelerated. The movement may follow a random or predetermined trajectory, such as a pattern, an array or a matrix. The movement of the at least one magnetic field generating device may be adjusted by an operator following the patient's needs. Exemplary embodiments of mechanisms enabling dynamic movement of the magnetic field generating device are described below.

The magnetic field generating device may be movable. The movement of the magnetic field generating device may be translational and/or rotational to provide various orientations of the magnetic field generation device within the magnetic stimulation device to improve targeting of the target biological structure or defocusing the peak of magnetic flux density. The translational movement may be according to at least one axis, more preferably according to at least two axes for providing movement in e.g. horizontal plane i.e. according to X and/or Y axes of CCS, or in all three axes of CCS for providing movement in a horizontal plane and also elevation adjustable to correspond with anatomical structures of the patient.

The movement of the magnetic field generating device may be also rotational around at least one axis, more preferably around two axes of a Cartesian coordinate system to improve targeting of the target biological structure.

In most designs, two different types of movement may be used for positioning and/or orienting the magnetic field generating device.

The movement may follow a predetermined trajectory in one plane. The movement may follow a grid pattern by scanning movement of the magnetic field generating device. The scanning movement may cover large body area such as entire pelvic floor. In an alternative embodiment the magnetic field generating device may adjust the position and/or the orientation during the treatment to dynamically stimulate different target biological structure and stimulate a greater area and/or volume.

A positioning mechanism for moving and/or orienting the magnetic field generating device may be used. The positioning mechanism may be an open or closed kinematic chain including at least one degree of freedom. In other embodiments the positioning mechanism may include: at least two degrees of freedom, e.g. two translational, two rotational, or one translational and one rotational around axis of translation; at least three degrees of freedom, e.g. three translational, or two translational and one rotational; or at least four degrees of freedom, e.g. three translational and one rotational or two translational and two rotational. In an alternative embodiment the positioning mechanism may include five degrees of freedom, e.g. three translational and two rotational. In all embodiments the degree of freedom providing elevation of the magnetic field generating device may be reduced and the elevation may be replaced by varying the amplitude of the magnetic flux density.

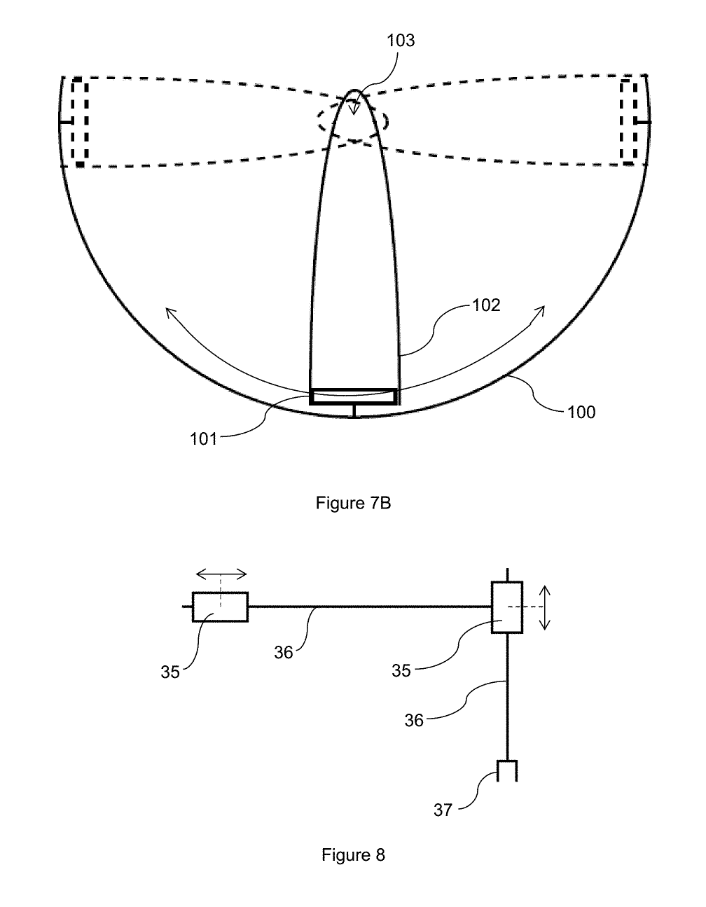

FIG. 8 illustrates a kinematic scheme of a positioning mechanism enabling scanning movement of the magnetic field generating device. The positioning mechanism includes two actuators 35 enabling translational movement of the mechanical links 36. The actuators may preferably move the links in two perpendicular directions. The magnetic field generating device 37 is drawn as endpoint.

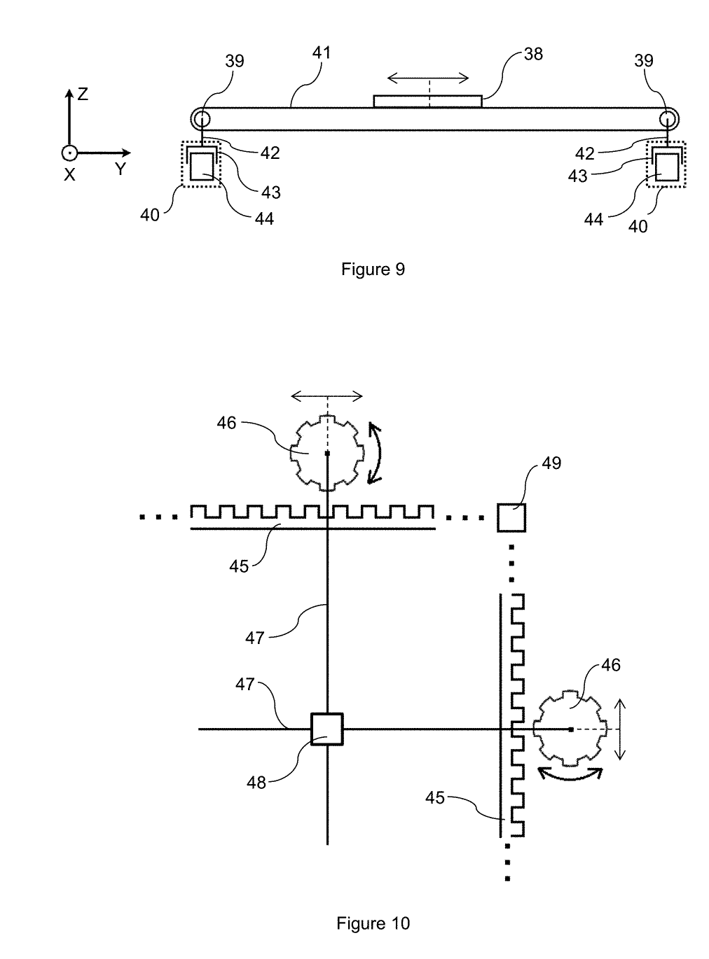

FIG. 9 illustrates another positioning mechanism enabling scanning movement of the magnetic field generating device 38. The positioning mechanism includes at least one rotational actuator 39 and at least one translational actuator 40. The actuator 39 propels a moveable member 41, e.g. a belt, in the Y-axis of CCS. The magnetic field generating device 38 is attached to the moveable member 41. The actuator 39 is connected via a link 42 with sliding member 43 moving in the X-axis of CCS. The movement of a sliding member 43 is propelled by the actuator 40. The movement of the sliding member 43 is guided by a guiding member 44 oriented according to X-axis of CCS.

FIG. 10 illustrates another positioning mechanism enabling scanning movement of the magnetic field generating device 38. The positioning mechanism includes at least two guiding members 45, two rotational actuators 46, two positioning links 47 connected together by sliding member 48, which is moveable according to each positioning link 47. The positioning mechanism may further include connecting member 49 for connecting guiding members 46 together, alternatively the guiding members 45 may be fixed together by e.g. welding, bonding (by glue or thermoplastic material) or any other permanent connection. The magnetic field generating device may be attached to the sliding member 48.

Alternatively at least two linear actuators may be used for constituting a positioning mechanism enabling scanning movement of the magnetic field generating device.

The elevation of the scanning mechanism may be enabled by additional actuator enabling movement in vertical direction, e.g. linear actuator or worm drive.

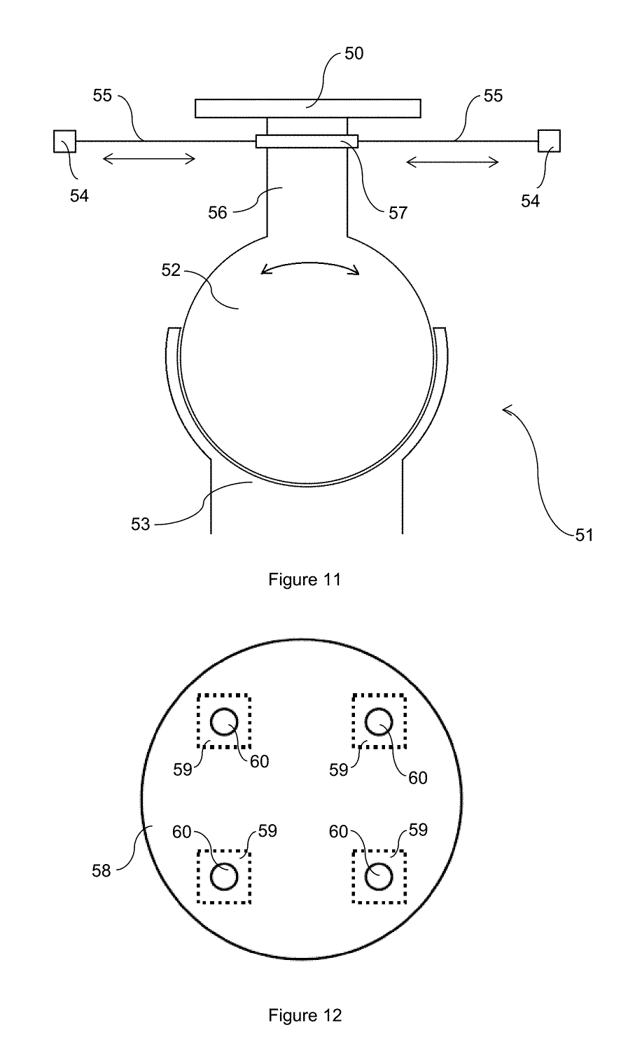

FIG. 11 illustrates cross-sectional view of an exemplary positioning mechanism enabling tilting of magnetic field generating device 50. The tilting movement is enabled by a joint mechanism 51. The joint mechanism 51 includes rotating member 52 which is housed in guiding member 53. Tilting of magnetic field generation device 50 in one direction may be enabled by cylindrical rotating member 52 preferably encased in U-shape guiding member 53. Tilting in a plurality of directions may be enabled by ball rotating member encased in a hollow ball guiding member 53. The movement of the magnetic field generating device 50 may be provided by at least one actuator 54 via link 55 connected to a shaft 56. The link 55 is attached to connecting member 57 which is moveable with respect to the shaft 56. The magnetic field generating device 50 may be preferably attached to the shaft 56 above the connecting member 57. The link 55 may alternatively include a curved member with a hollow part for guiding the movement of the shaft 56. In alternative embodiment the shaft may be below the rotating member and the magnetic field generating device may be attached directly to the rotating member.

Alternatively the positioning mechanism enables tilting of a magnetic field generating device via a mechanism as used in a gyroscopic joystick.

In an alternative embodiment the positioning mechanism may correspond with the anatomical shape of pelvic floor. The endpoint trajectory may move on or over a spherical surface.

The magnetic stimulation device may include at least one feedback information system for improving effectiveness and/or shortening duration of the treatment.

The feedback information may be provided by determining an active response for stimulation, e.g. at least partial muscle contraction. The at least partial muscle contraction causes dynamic forces. The dynamic forces caused by the at least partial muscle contraction may be determined by at least one sensor preferably placed beneath the patient, more preferably a plurality of sensors may be used as well. Following the feedback information the magnetic stimulation device may adjust the position and/or orientation of the magnetic field generating device with respect to the patient to improve the effectiveness of the treatment.

The feedback may be determined by at least one force sensor, e.g. load cell, placed below the magnetic stimulation device. More preferably a plurality of force sensors may be used, e.g. at least two, three or four force sensors. FIG. 12 is a bottom view of the magnetic stimulation device 58 showing four force sensors 59, e.g. weight sensors. The force sensors 59 are under each leg 60 of the magnetic stimulation device 58. Alternatively, at least one force sensor may be placed within the leg of the magnetic stimulation device where the sensor is unimpeded by the magnetic field generated by magnetic field generating device. In an alternative embodiment various sensors may be used for determining the feedback, exemplary suitable sensors and their application may be found in U.S. Pat. No. 7,030,764.

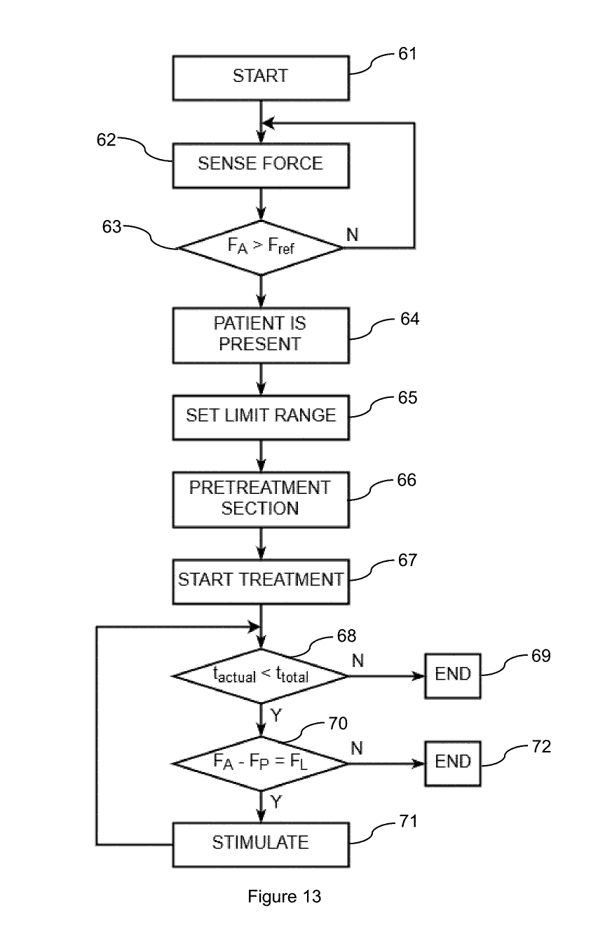

The at least one force sensor 59 may determine whether the patient is present on the magnetic stimulation device. Following this feedback the treatment may be automatically started. Such an application may be illustrated by the following exemplary embodiment described in FIG. 13. The at least one force sensor may be calibrated to reference value (F_{ref}) exerted by the magnetic stimulation device. In step 61, the magnetic stimulation device may be turned on. In step 62 the exerted force is sensed by at least one force sensor. In next step 63 the magnetic stimulation device examines whether the actual value (F_{A}) is greater than reference value (F_{ref}). If actual value (F_{A}) is not greater than reference value (F_{ref}) then the magnetic stimulation device may determine that the patient is not on the seat (not shown). If the actual value (F_{A}) is greater than reference value (F_{ref}) then the magnetic stimulation device may evaluate that the patient sits on the magnetic stimulation device 64.

As soon as the patient sits on the magnetic stimulation device a limit range of difference between actually measured value (F_{A}) and previously measured value (F_{P}) may be set in step 65. The limit range may be used for preventing incorrect ceasing of the treatment. The limit range (F_{L}) may be set automatically or manually. Automatically set limit range may be e.g. a preset value, or a percentage of the weight of the patient. In an exemplary embodiment the limit range may be at least 1 or more percent, e.g. 5, 10 or 15 percent. In an alternative embodiment the limit range may be preset by and adjusted by the operator.

In next step 66, at least one pretreatment section may be started. In an exemplary embodiment the pretreatment section may include positioning of the patient to correct treatment position, targeting the target biological structure, or determining optimal value of magnetic flux density for treatment which may be adjusted following the patient's needs. All the pretreatment sections may be processed automatically by the magnetic stimulation device and may be adjusted by the operator, or they may be processed manually by the operator. Afterwards the treatment may be started (step 67).

Afterwards actual treatment time (t_{actual}) may be compared to total treatment time (tₜₒₜₐₗ) in step 68. If the actual treatment time is at least equal to total treatment time then the treatment is stopped (step 69). If the actual treatment time (t_{actual}) is smaller than total treatment time (tₜₒₜₐₗ) the treatment may continue.

Then in step 70 is examined whether the difference of the actual value (F_{A}) and previously measured value (F_{P}) is within the limit range (F_{L}). If the difference between actual value (F_{A}) and previously measured value (F_{P}) is within limit range (F_{L}) then the magnetic stimulation device may determine that the patient remains in the magnetic stimulation device and the treatment continues by step 71.

If the difference between actual value (F_{A}) and previously measured value (F_{P}) is out of the limit range (F_{L}) then the treatment may be ceased in step 72 and error notification for the operator may be generated by the magnetic stimulation device in a human perceptible form, e.g. by mechanical and/or electromagnetic apparatus, such as audibly perceptible notification (e.g. beep) or visually perceptible notification (flashing light, color change etc.).

The routine may run continuously or in discrete time. Alternatively, the routine may run in predetermined time during the treatment, e.g. repeated in cycles lasting 2, 5 or 10 seconds.

In the case of application of the plurality of force sensors 59 an approximate position of patient's center of gravity may be determined.

Following the center of gravity position the magnetic field generating device may be automatically positioned and/or oriented to provide most effective treatment for the patient. Patient position may be approximated via virtual model of standardized patient using position of the patient's center of gravity position, alternatively various additionally patient parameters may be used, e.g. weight, height or BMI. Alternatively, the treatment may be automatically started and/or stopped following the position of the center of gravity.

If the position of center of gravity is within predetermined distance from the edge of the seating portion then the incorrect patient position may be determined and the patient may be repositioned. The notification concerning this fact may be generated for the operator by the magnetic stimulation device in a human perceptible form, e.g. by mechanical and/or electromagnetic apparatus, such as audibly perceptible notification (e.g. beep) or visually perceptible notification (flashing light, color change etc.). The repositioning may be done automatically and/or manually influenced by the distance and/or the patient's state.

The feedback may be determined by at least one image sensor, preferably video, photographic or IR sensor. Referring to FIG. 1 the image sensor 73 may be placed within proximity of the magnetic stimulation device to monitor the at least treated part of the patient. The image sensor may be placed in the room in a location enabling t monitoring at least the treated area of the patient. Alternatively, the image sensor may be integral part of the magnetic stimulation device. The signal from the at least one image sensor may be processed by processing unit to determine the contour of the at least treated area. Afterwards the position and/or orientation of the magnetic field generation device may be adjusted to improve the effectiveness of the treatment.

Alternatively the image sensor may be replaced by a distance sensor, e.g. light based sensors such as laser sensor, or mechanical wave based sensor such as ultrasound sensor.

Alternatively the patient position may be determined by a position determining system. The position determining system may include at least one reference marker, but more preferably a plurality of reference markers which may be attached to a patient to obtain the precise position of the patient following the position of the reference markers.

The patient position may be determined via a pressure sensitive layer 74 placed beneath the patient, preferably on the seating portion of the magnetic stimulation device. Alternatively the pressure sensitive layer may be a part of the seating portion. The pressure sensitive layer may be e.g. stripe-shaped, pad or the mattress. It may be made of a material enabling sensing the pressure changes or distributions, preferably using a biocompatible resilient material. The pressure sensitive layer may include at least one, more preferably a plurality of sensors which are able to determine the patient's position or location, and/or a change in the patient's position or location. The sensor may be represented by a force or weight sensor such as piezo-sensor, strain gauge or load cell, pressure sensor, temperature or optical sensor, capacitive sensor or sensor detecting changes, e.g. distance or velocity sensor, accelerometer or vibration sensor. A plurality of sensors may be preferably used in predefined locations, e.g. a grid or stripes.

A pressure sensitive layer may be placed on the seating portion of the magnetic stimulation device in the area of correct treatment position, e.g. in central area. The pressure sensitive layer may be a fluid filled and connected via a conduit with the pressure sensor external to magnetic field.

Alternatively, the pressure sensitive layer may include a plurality of cells in a predetermined pattern, e.g. an array or preferably a matrix. At least one sensor may be used to determine the pressure inside the pressure sensitive layer, a plurality of sensors may be preferably used. The pattern of cells may accurately determine the position of the patient by determining contact points.

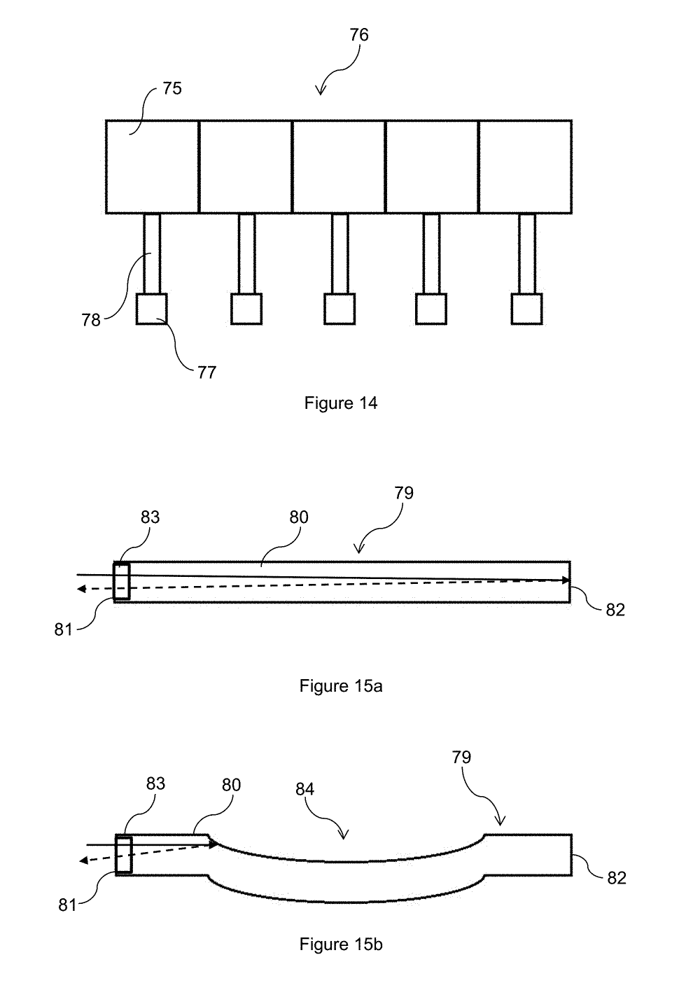

FIG. 14 illustrates an exemplary embodiment including a plurality of cells 75 in an array 76 using a plurality of pressure sensors 77. The cells are fluid-filled. The pressure of the fluid within the cells 75 is transmitted by tubes 78 to the sensors 77.

The pressure sensitive layer may be e.g. tube, film or sheet. It may be made of elastically deformable optic material which is at least partially reflective at the end. It may be oriented preferably transversally on the seating portion. The light may enter at one end of the optic material and propagate through the entire length of the optic material to the second end where it may be at least partially reflected. The intensity/energy of the at least partially reflected light may be determined. When patient sits on such pressure sensitive layer the optic path is shorter due to patient's weight. The attenuation is smaller and the intensity/energy of the at least partially reflected light is greater compared to the pressure sensitive layer when there is no patient on the seating portion. Alternatively, the time-of-flight may be used to determine the patient's position.

FIG. 15a illustrates an exemplary embodiment of a non-loaded optic sensor using reflected light. The optic sensor 79 includes light guiding member 80. The light may enter the light guiding member 80 at one end 81 of the light guiding member 80 and reflect at the opposite end 82 of the light guiding member 80. The intensity of the reflected light (dotted line) may be determined at the end 81 of the light guiding member 80, where the light entered the light guiding member, by a detector 83.

FIG. 15b illustrates the optic sensor 79 now under the load or weight of a patient. The patient's weight may create a hollow 84. The light enters the optic sensor 79 and may reflect from the hollow 84, hence the intensity of the reflected light (dotted line) may be greater compared to light intensity of the reflected light when the optic sensor is non-loaded.

Alternatively the magnetic stimulation device may include at least one optic band for determining the presence of the patient. Preferably a plurality of optic band may be used for determining the position of the patient.

The pressure sensitive layer may include at least one tube with constant fluid flow, preferably a plurality of fluid tubes may be used. Patient presence may be determined upon change of fluid flow. In preferred embodiment the fluid tube may be in a grid to determine contact points. Alternatively, the pressure sensitive layer may include a plurality of independent cells preferably in a predetermined pattern, e.g. a matrix. The contact cell may be determined by various manners using various approaches. For example the contact cell may be the cell which is loaded so the upper wall contacts the lower wall of the cell, it may be so called bottoming out. In this particular approach the contact cell may be determined by e.g. determining pressure change of cells in surroundings while the pressure in the contact cell remains constant. The pressure sensors may be preferably placed next to the seating portion to be unimpeded by the generated magnetic field.

The pressure sensitive layer may include at least one elastically deformable member which may be preferably oriented in the X-axis of CCS, in more preferred embodiment a plurality of elastically deformable members may be used. The at least one elastically deformable member may include a strain gauge on at least one end enabling determining the deformation of the at least one elastically deformable member in at least one direction, preferably in the Z-axis of CCS. In a preferred embodiment the deformation may be determined in a plurality of directions, most preferred in at least two orthogonal directions, e.g. in the Z-axis of CCS and at least one of X and Y axes of CCS. In an alternative embodiment inclinometers may be used instead of strain gauges.

The pressure sensitive layer may be rigid and it may include at least one accelerometer in at least one location unimpeded by magnetic field. One-axis, more at least two-axis, most preferably three-axis accelerometer may be used. The accelerometer may be preferably oriented in vertical direction. The accelerometer may be preferably placed in at least one edge of the seating portion, more preferably in at least two opposite edges of the seating portion or more accelerometers may be used.

All the above recited feedback methods may be used for determining the active response. The feedback information and/or signal may be processed by a processing unit of the magnetic stimulation device. Using the feedback, the position and/or orientation of the magnetic field generating device may be adjusted automatically and/or manually.

A patient may be stimulated by at least one pretreatment sequence prior to the treatment. The pretreatment sequence is not intended to treat the patient. The pretreatment sequence may be used for improving the effectiveness of the treatment by e.g. setting the magnetic field generating device to an appropriate position and/or determining the appropriate magnetic flux density for the patient. Both pretreatment sequences may be controlled by processing unit of the magnetic stimulation device and may be influenced by the feedback information.

The target biological structure may be stimulated by a pretreatment sequence for placing the magnetic field generating device in appropriate position to treat the target biological structure providing the greatest effect for the patient.

The appropriate position may be found by using stimulation of constant treatment parameters, e.g. repetition rate, magnetic flux density or impulse duration, while the magnetic field generating device scans the target biological structure. The time duration may be up to several minutes, more preferably in the range of 1 to 60 seconds, most preferably up to 30 seconds. The appropriate position may be found by firing at least two pulses, preferably at least 10 pulses, more preferably at least 50 pulses, most preferably at least 100 pulses or up to 500 pulses.

The appropriate position may be determined via registering the induced biological response, e.g. visually observed, perceived by the patient or detected by the feedback sensing device. The greatest effect for the patient may be achieved e.g. by stimulation of motor point, or by stimulation in such a position of the magnetic field generating device where the biological response is the weakest. The weakest biological response may correspond with the stimulation of weakened muscle which needs to be strengthened.

The appropriate position of the magnetic field generating device may be manually determined by the operator of the magnetic stimulation device while the operator observes the biological response of the target biological structure.

Alternatively, the patient may determine the appropriate position of the magnetic field generating device by using control apparatus following the perception of the stimulation. The control apparatus may include e.g. a lever mechanism, a joystick or control buttons linked to a seat actuator. Alternatively the control apparatus may adjust the position and/or orientation of the magnetic field generating device.

In an alternative embodiment the appropriate position of the magnetic field generating device may be set automatically by positioning mechanism following the feedback information.

The target biological structure may be stimulated by another pretreatment sequence including a plurality of pulses of different repetition rates. Following the pretreatment sequence an appropriate magnetic flux density may be determined for the treatment. The pretreatment sequence includes at least one repetition rate, more preferably at least two different repetition rates. The complete pretreatment sequence may last up to 120 seconds, more preferably in the range of 1 to 60 seconds, most preferably around 30 seconds.

The pretreatment sequence may include one repetition rate including at least one pulse, more preferably a plurality of pulses, e.g. at least two pulses, more preferably at least 5 pulses, even more preferably at least 10 or more pulses. The plurality of pulses is called a train. The magnetic flux density may be adjusted by an operator during the pretreatment sequence to provide the patient the appropriate treatment.

Alternatively, the pretreatment sequence may include a plurality of trains of different repetition rates. The repetition rate of first train may be the lowest repetition rate of the treatment. The repetition rate of second train may be the highest repetition rate of the treatment.

The magnetic flux density may be adjusted by an operator during the pretreatment sequence. The magnetic flux density of the trains may be the same for at least two trains.

In exemplary embodiment appropriate treatment parameters may be determined by the operator and/or the patient following the patient's needs.

Alternatively, the appropriate treatment parameters may be determined automatically by the magnetic stimulation device influenced the feedback information.

All the above recited methods and embodiments may be used for optimizing the treatment. The term optimizing treatment includes adjusting the position and/or orientation of the magnetic field generating device and/or treatment parameters. In preferred embodiment the treatment optimizing may be influenced feedback.

Novel systems and methods have been described. The invention should be interpreted in the broadest sense. Various changes and substitutions may be made of course without departing from the spirit and scope of the invention. The invention, therefore, should not be limited, except by the following claims and their equivalents.

It is to be understood that the method is not limited to the particular applications and that the method may be practiced or carried out in various ways.

### ICORPORATION BY REFERENCE

The following applications are incorporated herein by reference:

No. 62/357,679 filed Jul. 1, 2016 and now pending; Ser. No. 15/178,455 filed Jun. 9, 2016 and now pending; Ser. No. 15/151,012 filed May 10, 2016 and now pending; Ser. No. 15/099,274 filed Apr. 14, 2016 and now pending; Ser. No. 15/073,318 filed Mar. 24, 2015 and now pending; Ser. No. 14/951,093 filed Nov. 24, 2015 and now pending; Ser. No. 14/926,365 filed Oct. 29, 2015 and now pending; and Ser. No. 14/789,658 filed Jul. 1, 2015 and now pending; Ser. No. 14/873,110 filed Oct. 1, 2015 and now pending; No. U.S. patent application Ser. No. 14/789,156 filed Jul. 1, 2015 and pending.

### LIST OF REFERENCE NUMBERS

1 energy source
2 switching device
3 energy storage device
4 magnetic field generating device
5 protective circuitry
6 magnetic stimulation device
7 seating portion
8 cover
9 actuator
10 pivot axis
11 back rest
12 arm rest
13 resting apparatus
14 sensor
15 patient supporting apparatus
16 seating portion
17 recess of the seating portion for inserting the external applicator
18 external applicator
19 locking mechanism
20 locking mechanism
21 seating portion
22 recess of the locking mechanism in seating portion
23 resilient member
24 latching member
25 external applicator
26 recess of the locking mechanism in external applicator
27 magnetic field generating device
28 inner diameter
29 outer diameter
30 shape adjusting member
31 magnetic field generating device
32 moveable member
33 guiding member
34 rotating member
100 guiding member
101 magnetic field generating device
102 magnetic field
103 focus point of magnetic field
35 actuator
36 link
37 magnetic field generating device
38 magnetic field generating device
39 rotational actuator
40 translational actuator
41 moveable member
42 link
43 sliding member
44 guiding member
45 guiding member
46 rotational actuator
47 positioning link
48 sliding member
49 connecting member
50 magnetic field generating device
51 joint mechanism
52 rotating member
53 guiding member
54 actuator
55 link
56 shaft
57 connecting member
58 magnetic stimulation device
59 force sensor
60 leg of the magnetic stimulation device
61 start
62 force sensing
63 comparing sensed force to reference
64 determining patient's presence
65 setting limit range
66 pretreatment section
67 start of the treatment
68 comparing actual time to total treatment time
69 end
70 determining whether the sensed force is within limits
71 stimulation
72 end
73 image sensor
74 pressure sensitive layer
75 cell
76 array
77 pressure sensor
78 tube
79 optic sensor
80 light guiding member
81 end of light guiding member where the light enters
82 end of light guiding member where the light is reflected
83 detector
84 hollow

1-87. (canceled)

88. A magnetic stimulation device for treating a patient by a time-varying magnetic field including a connection to an energy source, a switching device, an energy storage device, a magnetic field generating device and a patient support including a bed or a seating portion of a chair,
the magnetic field generating device is in or below the patient support in proximity of a patient's pelvis;
the switch is configured to enable discharging the energy storage device to the magnetic field generating device in order to generate the time-varying magnetic field;
wherein the device includes a positioning mechanism for translational and/or rotational movement of the magnetic field generating device with respect to the patient and the patient support.

89. A magnetic stimulation device for treating a patient by a time-varying magnetic field including a connection to an energy source, a switching device, an energy storage device, a magnetic field generating device and a patient support including a bed or a seating portion of a chair,
the magnetic field generating device is in or below the patient support in proximity of a patient's pelvis;
the switching device is electrically connected to the energy storage device to enable a controlled discharge to the magnetic field generating device in order to generate the time-varying magnetic field;
wherein the magnetic stimulation device further includes a front resting apparatus for maintaining the patient in a bent or reclined position in a range of −45° to 90° with respect to vertical.

90. The device of claim 89 wherein the front resting apparatus is moveable separately from the magnetic treatment device.

91. The device of claim 89 wherein the front resting apparatus includes a sensor for measuring a distance of the front resting apparatus from the magnetic stimulation device and/or an inclination of the front resting apparatus.

92. The device of claim 91 wherein a distance of the front resting apparatus from the magnetic stimulation device as measured by the distance sensor is evaluated by a processing unit in order to maintain the patient in safe position.

93. The device of claim 89 wherein the front resting apparatus is adapted to be in contact with a patient's body part including a hand, a forearm or chest of the patient.

94. A magnetic stimulation device for treating a patient by a time-varying magnetic field including a connection to an energy source, a control unit, a switching device, an energy storage device, a magnetic field generating device and a patient support including a bed or a chair:
wherein the magnetic field generating device is in the patient support and/or beneath the patient support of the magnetic stimulation device; and
wherein the magnetic field generating device is within proximity and beneath a pelvic floor of the patient;
wherein the control unit is configured to control the switching device;
wherein the energy storage device is electrically connected with the magnetic field generating device to be discharged to the magnetic field generating device which generates the time-varying magnetic field with a magnetic flux density in a range of 0.1 to 7 T, a repetition rate in a range of 1 to 700 Hz, an impulse duration in a range of 10 to 1000 μs and with a treatment duty cycle over 10%;
wherein the magnetic field generating device includes a litz-wire;
wherein the magnetic field generating is cooled.

95. The magnetic stimulation device of claim 94 wherein the magnetic stimulation device further includes a sensor which determines an operation parameter of the magnetic stimulation device including voltage, current or magnetic flux density.

96. The magnetic stimulation device of claim 95 wherein the control unit is configured to process a determined operation parameter value and wherein the control unit is configured to determine an unintended event including a metal object within proximity of the magnetic field generating device and/or a hardware error of the magnetic stimulation device and wherein the control unit is configured to cease and/or to provide a notification to an operator if the unintended event is determined by the control unit.

97. The magnetic stimulation device of claim 95 wherein the control unit is configured to process a determined operation parameter value and wherein the control unit is configured to provide a notification including a maximal value of at least one treatment parameter including the magnetic flux density, the repetition rate, the impulse duration and a treatment duration to an operator in a human perceptible form based on the operation parameter value measured by the sensor.

98. The magnetic stimulation device of claim 95 wherein the control unit is configured to process a determined operation parameter value and wherein the control unit is configured to evaluate the determined operation parameter value in order to provide energy to the energy storage device.

99. The magnetic stimulation device of claim 94 wherein the magnetic field generating device includes a conductor of diameter less than 0.5 mm.

100. The magnetic stimulation device of claim 94 wherein the magnetic stimulation devices includes a blower on a circumference of the magnetic field generating device.

101. The magnetic stimulation device of claim 94 wherein the magnetic field generating device is cooled by a cooling media parallel with the magnetic field generating device.

102. The magnetic stimulation device of claim 94 wherein the magnetic field generating device is cooled by a cooling media flow over at least upper and lower side.

103. The magnetic stimulation device of claim 94 wherein the magnetic stimulation device further includes a device which generates electromagnetic waves which includes a high-frequency generator, a balun transformer, a transmatch and an electrode;
wherein the electrode is configured to generate the electromagnetic waves with a frequency in a range of 1 MHz to 3 GHz; and
wherein the electrode is in the patient support and within proximity and/or beneath a pelvic floor of the patient.

104. A magnetic stimulation device for treating a patient by a time-varying magnetic field including a connection to an energy source, a switching device, an energy storage device, a magnetic field generating device and a patient support including a bed or a chair:
wherein the magnetic field generating device is in the patient support and/or beneath the patient support of the magnetic stimulation device; and
wherein the magnetic field generating device is within proximity and beneath a pelvic floor of the patient;
wherein the energy storage device is electrically connected with the magnetic field generating device to be discharged to the magnetic field generating device which generates the time-varying magnetic field with a magnetic flux density in a range of 0.1 to 7 T, a repetition rate in a range of 1 to 700 Hz, an impulse duration in a range of 10 to 1000 μs and with a treatment duty cycle over 10%;
wherein the magnetic field generating device is cooled;
wherein the magnetic stimulation device enables generating magnetic pulses with a variable repetition rate and/or a variable magnetic flux density during one treatment;
wherein the magnetic stimulation device enables assembling magnetic pulses into a sinusoidal, triangular, saw-tooth, trapezoidal or exponential shape.

105. The magnetic stimulation device of claim 104 wherein the magnetic field generating device includes a litz-wire.

106. The magnetic stimulation device of claim 105 wherein the magnetic field generating device include a conductor of diameter less than 3 mm.

107. The magnetic stimulation device of claim 104 wherein the magnetic stimulation devices includes a blower on a circumference of the magnetic field generating device.

108. The magnetic stimulation device of claim 104 wherein the magnetic field generating device is cooled by a cooling media parallel with the magnetic field generating device.

109. The magnetic stimulation device of claim 104 wherein the magnetic field generating device is cooled by a cooling media flow over at least upper and lower side.

110. The magnetic stimulation device of claim 104 wherein the magnetic field generating device is in a serial connection with the energy storage device.

111. The magnetic stimulation device of claim 110 wherein the switching device is in a parallel connection with the serial connection of the magnetic field generating device and the energy storage device.

112. The magnetic stimulation device of claim 104 wherein the switching device is in a parallel to the energy source in order to enable controlled shorting of the energy source.

113. The magnetic stimulation device of claim 104 wherein the magnetic stimulation device further includes sensor which determines an operation parameter including a voltage, a current or a magnetic flux density.

114. The magnetic stimulation device of claim 113 wherein an unintended event is determined by a control unit based on information from the sensor determining the operation parameter;
wherein the unintended event includes a proximity of a metal object or a hardware error of the magnetic stimulation device.

115. The magnetic stimulation device of claim 114 wherein the control unit is configured to cease a treatment if the unintended event is determined.

116. The magnetic stimulation device of claim 113 wherein a control unit is configured to process a determined operation parameter value and wherein the control unit is configured to provide a notification including a maximal value of at least one treatment parameter including the magnetic flux density, the repetition rate, the impulse duration and a treatment duration to an operator in a human perceptible form based on operation parameter value measured by the sensor in order to maintain a casing of the magnetic field generating device up to 43° C.

117. The magnetic stimulation device of claim 104 wherein the magnetic stimulation device further includes a device which generates electromagnetic waves which includes high-frequency generator, a balun transformer, a transmatch and an electrode
wherein the electrode is configured to generate the electromagnetic waves with a frequency in a range of 1 MHz to 3 GHz; and
wherein the electrode is in the patient support and within proximity and/or beneath a pelvic floor of the patient.
